(19) United States
(12) Patent Application Publication
(10) Pub. No.: US 2023/0026586 A1
(43) Pub. Date: Jan. 26, 2023

(54) System and Method for a Microfluidic Jet Generation from a Compact Device

(71) Applicant: ECOLE POLYTECHNIQUE FEDERALE DE LAUSANNE (EPFL), Lausanne (CH)

(72) Inventors: Jan Krizek, Renens (CH); Christophe Moser, Lausanne (CH); Frédéric De Goumoëns, Pasadena, CA (US)

(21) Appl. No.: 17/783,690

(22) PCT Filed: Jan. 27, 2021
(86) PCT No.: PCT/IB2021/050625

§ 371 Date: Jun. 9, 2022

(30) Foreign Application Priority Data
Jan. 28, 2020 (IB) PCT/IB2020/050650

(51) Int. Cl. A61M 5/30; A61B 18/22; A61M 5/20

(52) U.S. Cl. CPC A61M 5/30; A61B 18/22; A61M 5/2046; A61B 2018/2211

(57) ABSTRACT

The invention discloses systems and methods for generation of microfluidic jets providing a tool for very precise and localized delivery of e.g., medicaments. The proposed solution overcomes shortcomings related to miniaturization of a jet injection technology by implementing laser energy as a driving mechanism and optical fibers for its delivery. Solving the step of miniaturization can allow building new tools compatible with minimally invasive surgical techniques, high parallelization of jet injection units or design of new ergonomic injection devices.

### FIELD OF THE INVENTION

The present invention relates to a system and a method for generation of microfluidic jets. More particularly, the present invention provides a device and a method for controlled delivery of small amounts of liquids, e.g., medicaments, using microjets generated from a compact system.

### BACKGROUND ART

The most common route for medicaments to a human body has been via oral ingestion of pills. The ongoing trend in medicine is to mitigate side effects and thus to target specific body locations. There is also a variety of advanced therapies requiring specific localization with precise dosing and consequently, systematic administration is not appropriate. Needle injection is one of the classical routes for localized drug delivery. But the current challenge is not only in the system miniaturization but also in the precision of the drug dose delivery and its spatial localization. Jet injection technique is a promising candidate to solve dosing and localization problem, however, in the present art, there is a lack of solutions providing compact systems allowing operation in physically restricted areas or schemes capable of parallelization of the delivery process from multiple units such as is proposed in US 2004/0260234 A1.

In jet injection devices, the liquid is pushed out of the nozzle forming a thin jet, which is powerful enough to penetrate through a tissue. The actuation scheme is commonly based on a piezoactuator, loaded spring, gas cartridge, voice coil actuator or electrical discharge. These actuation mechanisms are bulky and do not allow implementation as a compact minimally invasive device. Even if miniaturization could be performed it would pose a safety concern about bringing high voltages or high-pressure elements inside the human body. A method for the generation of jet in a liquid environment by electric discharge is described in U.S. Pat. No. 6,913,605 B2. This document mentions also the laser actuation but does not propose any solution.

A convenient method for a pressure and consequent jet generation is by using lasers. Laser radiation causes fast vaporization of the fluid and consequent bubble expansion generates driving pressure force for the jet. Documents U.S. Pat. No. 8,905,966 B2, US 2015/0265770 A1, US 2012/0215098 A1, U.S. Pat. No. 9,351,817 B1 describe systems which consist of two separate compartments: one for the light absorption driving fluid and the second is the channel for the drug content, the pressure is then transmitted by an elastic membrane. As the mechanical energy is mediated through the membrane it requires a significant laser energy pulse (>100 mJ) to produce liquid jets of sufficient power. Another laser jet generating apparatus has been proposed as a dissection device U.S. Pat. No. 7,815,632 B2, U.S. Pat. No. 7,740,626 B2. Here, the actuation scheme is realized by a high energy nano second light pulse delivered by an optical fiber directly to the ejected fluid. The proposed solution relies on a high energy laser source generating nano-second pulses of significant energy (>50 mJ) which exclude the use of thin optical fibers (<500 μm) due to potentially high energy densities and limiting laser induced damage threshold. Using lower energy actuation (<10 mJ) in the aforementioned schemes would generate jet of insufficient power to penetrate into a broad range of body tissue. High energy generation requirement necessitate costly and large laser systems. Therefore, there is a need to augment the effectiveness of energy translation between a laser radiation and a liquid jet. One solution was proposed in WO 2011/144496 A1 by using physical phenomena known as a flow focusing effect (Tagawa et. al. https://doi.org/10.1103/PhysRevX.2.031002.). Sub-nozzle diameter and very fast jets can be generated while using a scheme where a liquid/air interface at the nozzle opening has at least partially concave shape. Thanks to an additional acceleration by the flow focusing effect, their system uses nano-second optical pulses of relatively smaller energy (<3 mJ) focused by a microscope objective from the side of the transparent nozzle. The systems with a side focusing lens are not compact and complicate implementation into hand-held or minimally invasive device. Another solution for improvement of the delivery efficiency is proposed in “Needle assisted jet injection” U.S. Pat. No. 7,776,015 B2, A short needle is used to pierce the first layer of the skin and thus lowers the mechanical barrier which reduces a jet power needed to achieve the desired location.

Using optical fibers or other waveguides unfolds the potential of lasers as a driving mechanism. The fact that the energy can be remotely generated in a large laser system and then delivered by means of very thin and flexible fibers is convenient for miniaturization of the jet delivery device. Flexibility adds opportunity to make devices compatible with minimally invasive surgical techniques, such as for example endoscopy or others. A specific dynamics of the generation allows taking advantage of the flow focusing phenomena such as described by Tagawa et. al. (https://doi.10.1103/PhysRevX.2.031002). Moreover, the compactness of the optical fiber allows to design new schemes for needle-assisted fluid delivery.

Accordingly, the present invention aims to provide a solution to miniaturization of the jet injection technology.

### SUMMARY OF THE INVENTION

In a first aspect, the invention provides a method for jet ejection of a fluid towards a substrate, comprising providing a nozzle filled inside with the fluid, positioning a fiber source of pulsed radiation inside the nozzle in direct contact with the fluid, the fiber source comprising an optical fiber. The fluid is configured to absorb at least a part of the radiation. The method further comprises generating a bubble inside the nozzle by absorbing a pulse of the pulsed radiation in a first portion of the fluid, thereby vaporizing the fluid into the bubble, pushing a second portion of the fluid out of an opening at an extremity of the nozzle by an effect of an expansion of the bubble, whereby the extremity is directed to the substrate, thereby enabling the jet ejection of the second portion of fluid, and an intensity of the pulse being configured to be inferior to a radiation-induced threshold of a material comprised in the fiber source.

In a preferred embodiment, the method further comprises adding an absorbing additive to the fluid, the absorbing additive being configured to improve the absorption of the pulse in the fluid and the vaporization thereof.

In another preferred embodiment, the source of laser pulses emits in the spectrum which is intrinsically absorbed by the liquid.

In another embodiment, interaction of the short pulse (<100 ns) laser radiation with the liquid can happen under heat and stress confined conditions which mitigates degrading effects on the sensitive formulations.

In a further preferred embodiment, the method further comprises integrating a focusing mechanism in the fiber source configured to focus radiation out of the optical fiber, thereby enabling an increase of the intensity of the pulse.

In a further preferred embodiment, the method further comprises positioning the extremity in a gaseous environment, and providing a layer of hydrophilic material at the opening, thereby enabling a formation of a concave meniscus directed to the inside of the nozzle, the concave meniscus defining an interface between the fluid and the gaseous environment, the concave meniscus being configured to center the jet ejection in the nozzle, and accelerate the jet ejection by a flow focusing effect.

In a further preferred embodiment, the method further comprises positioning the extremity in a liquid environment, introducing at the extremity a determined volume of gas to enable a controlled gas bubble creation between the substrate and an end of the fiber source, thereby enabling a formation of a concave meniscus directed to the inside of the tubular nozzle, the concave meniscus defining an interface between the fluid and the controlled gas bubble environment, the concave meniscus being configured to center the jet ejection in the nozzle, and accelerate the jet ejection by a flow focusing effect.

In a further preferred embodiment, the method further comprises configuring the intensity, an absorption of the pulse in the fluid, and the formation of the concave meniscus such to enable the second portion to penetrate into the substrate.

In a further preferred embodiment, the step of configuring is such to enable the second portion to pierce the substrate.

In a further preferred embodiment, the method further comprises configuring the intensity, an absorption of the pulse in the fluid, and the formation of the concave meniscus such to enable the second portion to be deposited on a top of the substrate.

In a further preferred embodiment, the substrate is a biological tissue.

In a further preferred embodiment, the method further comprises providing further nozzles filled inside with the fluid, and positioning further fiber sources of pulsed radiation respectively inside the corresponding nozzle in direct contact with the fluid, thereby enabling to concurrently generate the jet ejection for each nozzle.

In a second aspect, the invention provides a method for a needle assisted jet injection of a fluid in a substrate, comprising piercing the substrate with an injection needle, and applying pressure by means of a pressure generating mechanism, to the fluid at an entrance side of the injection needle to inject the fluid through the injection needle. The step of applying a pressure involves causing a rapid phase transition of a first portion of the fluid, thereby generating a bubble configured to expand and inject a second portion of fluid through the injection needle.

In a further preferred embodiment, the pressure generating mechanism comprises a pulsed radiation source.

In a further preferred embodiment, the pressure generating mechanism comprises an electrical discharge source.

In a further preferred embodiment, the pressure generating mechanism comprises a compartment configured to be filled by the fluid, and fluidically connected to the entrance side of the injection needle.

In a further preferred embodiment, the compartment further comprises a window transparent to the pulsed radiation source.

In a further preferred embodiment, the compartment further comprises a separation part configured to absorb the pulsed radiation and thereby lead to the rapid phase transition of the first portion of the fluid.

In a further preferred embodiment, the step of applying pressure is further configured to enable an adjustment of a depth in the substrate at which the fluid may be jet injected. The method further comprises generating a bubble inside the nozzle by absorbing a pulse of the pulsed radiation in a first portion the fluid, thereby vaporizing the fluid into the bubble, pushing a second portion of the fluid out of an opening at an extremity of the nozzle by an effect of an expansion of the bubble, whereby the extremity is directed to the substrate, thereby enabling the jet injection of the second portion of fluid, and an intensity of the pulse being configured to be inferior to a radiation-induced threshold of a material comprised in the fiber source.

In a third aspect, the invention provides a method for a needle assisted jet injection of a first fluid in a substrate, comprising piercing the substrate with an injection needle, applying pressure by means of a pressure generating mechanism, to the first fluid at an entrance side of the injection needle to inject the first fluid through the injection needle, and providing a first compartment configured to contain the first fluid. The step of applying pressure further comprises providing a second compartment configured to contain a second fluid, and causing a rapid phase transition of a portion of the second fluid, thereby generating a bubble configured to expand, apply pressure to the first compartment, and inject a part of the first fluid though the injection needle.

In a further preferred embodiment, the pressure generating mechanism comprises a pulsed radiation source.

In a further preferred embodiment, the pressure generating mechanism comprises an electrical discharge source.

In a fourth aspect, the invention provides a jet ejection device for jet ejection of a fluid towards an intended substrate, comprising a nozzle, a fiber source of pulsed radiation positioned inside the nozzle, the fiber source comprising an optical fiber, the nozzle being filled inside with the fluid, and the fiber source being in direct contact with the fluid. The fluid is further configured to absorb at least a part of the radiation and to generate a bubble inside the nozzle when a pulse of the pulsed radiation is absorbed in a first portion of the fluid and vaporizes the fluid into the bubble, whereby an effect of expansion of the bubble causes a second portion of the fluid to be pushed to an opening at an extremity of the nozzle directed to the intended substrate, thereby enabling the jet ejection of the second portion of fluid.

In a further preferred embodiment, the jet ejection device further comprises an absorbing additive for the fluid, configured to improve absorption of the pulse in the fluid and the vaporization thereof.

In a further preferred embodiment, the jet ejection device further comprises a focusing mechanism in the fiber source configured to focus radiation out of the optical fiber, thereby enabling an increase of the intensity of the pulse.

In a further preferred embodiment, the jet ejection device further comprises a layer of hydrophilic material at the opening, configured to enable a formation of a concave meniscus directed to the inside of the nozzle, the concave meniscus defining an interface between the fluid and a gaseous environment at the opening of the nozzle, the concave meniscus being configured to center the jet ejection in the nozzle and accelerate the jet ejection by a flow focusing effect.

In a further preferred embodiment, the jet ejection device further comprises a gas channel configured to deliver gas at the opening of the nozzle, and thereby to enable the formation of the concave meniscus.

In a further preferred embodiment, the jet ejection device further comprises a flexible tubing leading to the nozzle

In a fifth aspect, the invention provides a needle assisted jet injection device for injection of a fluid in an intended substrate, the device comprising an injection needle, having an entrance side through which fluid enters the injection needle, a fluid supply channel configured to supply the fluid, whereby the entrance side of the injection needle is attached to an output of the fluid supply channel, a pressure generating mechanism configured to apply pressure to the fluid at the entrance side of the injection needle. The pressure generating mechanism comprises a pulsed energy supply configured to deliver a pulse of energy in the fluid in the vicinity of the entrance side, thereby causing a rapid phase transition of a first portion of the fluid and generating a bubble configured to expand and inject a second portion of fluid through the injection needle.

In a further preferred embodiment of the needle assisted jet injection device the pulsed energy supply comprises a pulsed radiation source.

In a further preferred embodiment, the pulsed energy supply comprises an electrical discharge source.

In a sixth aspect, the invention provides a use of the jet ejection device as described herein above, to achieve any one of the following list:
injection of the fluid in the substrate,
depositing of the fluid on the substrate,
injection of the fluid through the substrate.

In a seventh aspect, the invention provides a use of the needle assisted jet injection device as described herein above, to achieve any one of the following list:
injection of the fluid in the substrate,
depositing of the fluid on the substrate,
injection of the fluid through the substrate.

In an eighth aspect, the invention provides a patterned arrangement of multiple nozzles such as described in the first aspect herein above.

In a further preferred embodiment, the patterned arrangement comprises a radiation splitting means configured to generate a plurality of partition of radiation, each partition corresponding to a respective nozzle of the multiple nozzles.

In a further preferred embodiment, the radiation splitting means is any one of the items in the list comprising at least a beam splitter, a lens microarray, a hologram, a fiber-based splitter, a fiber bundle system.

### BRIEF DESCRIPTION OF THE DRAWINGS

These and other features, aspects and advantages of the present invention will become better understood with regard to the following detailed description, appended claims and accompanying drawings where:

FIG. 1 contains a schematic of a compact system generating liquid microjet (107) in the gaseous environment (102) by a laser actuation (105) with a source having direct contact with ejected fluid by optical fiber (106), whereby in FIG. 1a the drawing depicts the status of the system before the liquid jet emerges, and in FIG. 1b the drawing depicts the status of the system after the liquid jet emerges;

FIG. 2 contains a schematic example of arrangement of multiple embodiments from FIG. 1 to concurrently generate multiple liquid microjets in the air environment;

FIG. 3 contains a schematic of a compact system to generate liquid microjet (308) in the liquid environment (301) with a gas channel (305) allowing bubble generation (306) at the channel opening, whereby in FIG. 3a the drawing depicts the status of the system before the liquid jet emerges, and in FIG. 3b: the drawing depicts the status of the system after the liquid jet emerges;

FIG. 4 contains a schematic depiction of the needle assisted (401) jet injection using a vapor bubble (405) pressure generation scheme (402) in the direct contact to a released fluid;

FIG. 5 contains a schematic example of arrangement of multiple systems from FIG. 4 to concurrently generate multiple liquid injections from the needle-assisted jet injectors;

FIG. 6 contains a schematic view of a needle assisted jet injection system using electromagnetic radiation (603) as a driving force for the liquid jet actuation while the source of the radiation is comparted from the fluid (606) by separation (601) transparent to the radiation; variations of the actuation scheme:

FIG. 6a: a source of radiation is delivered by an optical waveguide (602) and is further absorbed by the fluid (606);

FIG. 6b: radiation (609) is focused by e.g., lens (610) and passes through a transparent separation (601) and is further absorbed by the fluid (606);

FIG. 6c: source of radiation is delivered by an optical waveguide (602) and it is further absorbed by the solid-state light-absorbing material (611) coated on the transparent separation (601);

FIG. 6d: radiation (609) is focused by e.g., lens (610) and passes through a transparent separation (601) and it is absorbed by the solid-state light-absorbing material (611) coated on the transparent separation (601).

FIG. 6e: source of radiation is delivered by an optical waveguide (602) and is further absorbed by the solid-state light-absorbing material (611) coated on the transparent separation (601). Elastic coating of e.g., polyimide (612) contains the expanding metallic vapor within the blister; and

FIG. 6f: radiation (609) is focused by e.g., lens (610) and passes through a transparent separation (601) further it is absorbed by the solid-state light-absorbing material (611) coated on the transparent separation (601). Elastic coating of e.g., polyimide (612) contains the expanding metallic vapor within the blister;

FIG. 7 contains a schematic view of a needle assisted jet injection system using electromagnetic radiation as a driving force for the liquid jet actuation. The system comprises two separate compartments; one for the light absorption driving fluid (705) and the second is the channel for the fluid content (702), the pressure is then transmitted by an elastic membrane (704); wherein in FIG. 7a a source of radiation is delivered to the chamber with the driving fluid (705) by an optical waveguide (706); and in FIG. 7b the radiation (710) is focused by an optical focusing element (711) into the chamber containing the driving fluid (705);

FIG. 8 shows an example of the device described by the scheme of the FIG. 1;

FIG. 9 shows an example of the jet injection from the device depicted on the FIG. 8; and

FIG. 10 is a schematic view of a system with a flexible tubing.

### DETAILED DESCRIPTION

The present invention seeks to provide a solution to miniaturization of the jet injection technology by using optical energy as a driving force and delivery by means of optical fibers.

In one aspect of the invention, A fiber source of radiation, e.g., a fiber optics, is used to deliver the light energy into a nozzle filled with a fluid, whereby the fluid is configured to absorb radiation in a spectrum of the used radiation. The fiber source of radiation is in direct contact with the absorbing fluid. This enables to avoid any unwanted modulation by an other medium like a nozzle material or free space.

An intensity of the radiation is smaller than the laser-induced-damage-threshold (typically <5 GW/cm² for silica step-index fiber, but can be higher for hollow-core fibers or photonic crystal fiber) of an optical fiber in the fiber optics, but higher than a threshold for the vapor bubble generation in the fluid, which depends on a nature of the fluid. To secure this condition, appropriate absorbing additives may be added to the fluid. Conditions for the bubble cavitation can be also secured by the appropriate choice of the laser source in the near infra-red emitting spectrum which is the wavelength absorbed by water and other pharmaceutical solvents—this way additives can be avoided. Interaction of the short pulse (<100 ns) laser radiation with the liquid can happen under heat and stress confined conditions which mitigates degrading effects on the sensitive formulations. To facilitate coupling into the optical fiber it can be used tapered fiber with smaller and at the nozzle site. The nozzle can be accompanied by the mechanism allowing disposable use where the end section of the fiber can be reconnected by fiber connector.

Another solution to secure sufficient power for cavitation is the integration of a focusing mechanism on the end of the fiber such as for example, but not exclusively, a ball lens or a graded-index lens. Optical fibers with a smaller diameter, for example 50 μm, are more efficient in translation of the optical energy to the jet energy than thicker fibers, for example 200 μm.

In one embodiment a nozzle ending is in a gaseous environment and it is made from a hydrophilic material or a hydrophobic material equipped by a layer of the hydrophilic material at the opening, allowing the formation of a concave meniscus. The laser radiation is absorbed which leads to a rapid vaporization of the fluid and consequent generation of the bubble. As the bubble expands it pushes the fluid out of the channel. Thanks to the concave shape of the liquid/air interface the jet emerges from the center of the meniscus and it is further accelerated by the flow focusing effect.

In one example of the application, the jet velocity is sufficient to penetrate into the substrate such as for example, but not exclusively, biological tissue.

In another example of the application the jet velocity is sufficient to pierce through the substrate such as for example, but not exclusively, biological tissue.

In yet another example of the application the jet is deposited on the top of the substrate such as for example, but not exclusively, biological tissue. The aforementioned embodiment may be multiplied in single device to concurrently generate multiple injections.

According to another aspect of the present invention, the embodiment is modified for the operation in the liquid environment. In order to evoke the flow focusing effect and subsequent jet generation, a curved liquid/air interface is secured. That may be realized, for example, by additional gas inlet channel leading to the nozzle opening allowing a controlled gas bubble creation between the substrate and the optical fiber end. The absorption of laser radiation leads to the generation of the vapor bubble and thanks to the concave shape of the liquid/air interface formed by the gas bubble the jet emerges and is also further accelerated by the flow focusing effect.

In one example of the application, the jet velocity is sufficient to penetrate into the substrate such as for example, but not exclusively, biological tissue.

In another example of the application the jet velocity is sufficient to pierce through the substrate such as for example, but not exclusively, biological tissue.

In yet another example of the application the jet is deposited on the top of the substrate and does not penetrate in.

The aforementioned embodiment may be multiplied in single device to concurrently generate multiple injections. This can be achieved by splitting the radiation by optical element for example but not exclusively by a beam splitter, lens microarray, hologram, fiber-based splitter, or using a fiber bundle system. Each partition of radiation can serve as the source for the cavitation event. Parallel embodiment of nozzles can be arranged in a pattern. This may allow patterned deposition to target specific location of the tissue. Examples of the use of patterned arrangement may be in a localized distribution of dental anesthesia where specific arrangement can facilitate single tooth anesthesia or a needle-free based tattoo machine where this can allow new artistic expression extending a current single dot deposition to for example, but not exclusively multiple nozzles arranged in a line.

Another aspect of the invention relates to a needle assisted jet injection. The substrate, which may be a biological tissue, is partially or fully pierced by a short (<5 mm) and thin needle. This way a part of the mechanical barrier is overcome. The needle is equipped with a pressure generating mechanism. A pressure may be generated, for example, by the rapid phase transition initiated by laser radiation or an electrical discharge. An actuation mechanism may be in the direct contact with the fluid or it may propagate through the separation unit transparent to the radiation.

In one embodiment, radiation is delivered by the optical fiber.

In another embodiment radiation is focused by the free space optical element. Radiation may be absorbed directly by the fluid or by the solid-state light-absorbing material coated on the transparent separation unit being in contact with the fluid.

Both preceding embodiments lead to the generation of the vapor bubble which expands and pushes the fluid out of the needle into the substrate following a direction of the needle opening. The solid-state light-absorbing material coating may be further equipped by the elastic coating of for example, but not exclusively, polyimide. An additional layer may contain the expanding metallic vapor within but cause the layer deformation and creation of the expansion blister.

Each of the aforementioned embodiments may realize deposition in a certain depth depending on the energy of the actuation.

Each of the aforementioned embodiments may be multiplied in a single device to concurrently generate multiple injections. Parallel embodiment of nozzles can be arranged in a pattern.

In one example of the application, this scheme is used to enhance delivery from a microneedle array.

In yet another embodiment of a needle assisted jet injection, a system comprises two separate compartments: one for the highly absorptive driving fluid and the second is the channel for the functional fluid content for example drug, the pressure is then transmitted by an elastic membrane, or other movable unit. In one version of this embodiment a source of radiation is delivered by an optical waveguide.

In an other version of the embodiment the radiation is focused by an optical focusing element into the chamber containing the driving fluid.

In an other version of the embodiment the nozzle channel is tapered to achieve higher velocities.

In an other version of the embodiment the tubing leading to a nozzle is made from flexible material, for example but not exclusively Teflon.

The above-described applications are merely examples, and by no means exhaustive.

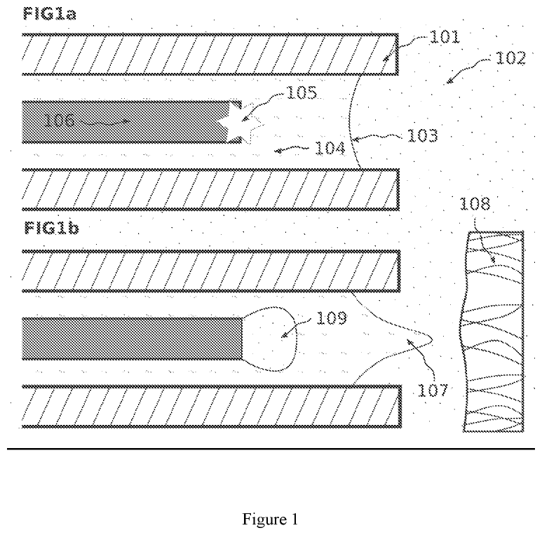

In FIG. 1a is a schematic depiction of a compact system consisting of a nozzle (101) made out of hydrophilic material or hydrophobic material equipped by a coating made with the hydrophilic material allowing a concave meniscus (103) to emerge between the fluid (104) and a gaseous environment (102). Laser pulse radiation (105) is delivered by an optical waveguide (106), e.g., an optical fiber, and it is in direct contact with the fluid (104) which is capable of absorption in the spectrum of the laser pulse radiation (105). FIG. 1b shows a drawing of a jet (107) emerging after the energy of the laser pulse radiation (105) is absorbed. Absorption leads to a rapid vaporization of the fluid (104) and generation of the bubble (109). As the bubble (109) expands, it pushes the liquid out of a channel of the nozzle (101). Thanks to the concave shape of the liquid/air interface (103) the jet (107) emerges from the center of the channel of the nozzle (101) and it is further accelerated by the flow focusing effect. In one example of the application, the jet velocity is sufficient to penetrate into a substrate (108). In another example of the application the jet velocity is sufficient to pierce through the substrate (108). In yet another example of the application the jet is deposited on the top of the substrate (108) and does not penetrate in.

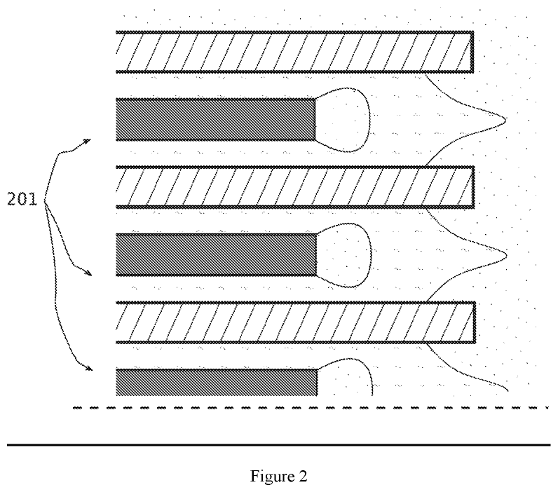

In FIG. 2 is a schematic example of an arrangement consisting of multiple systems described in the FIG. 1 (201) to concurrently generate multiple liquid microjets in the air environment.

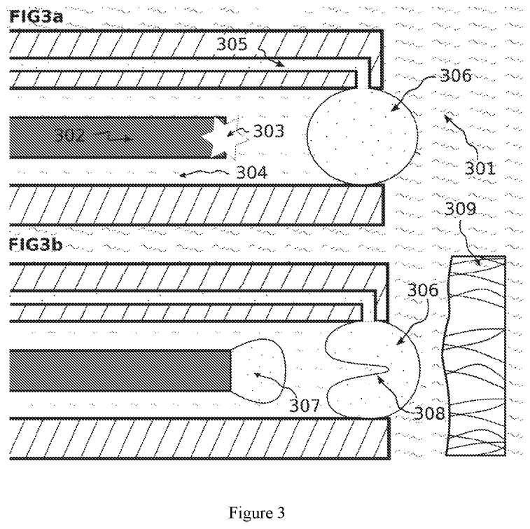

In FIG. 3 is a schematic drawing of a compact system adjusted for the generation of the liquid microjet (308) in the liquid environment (301). In order to evoke the flow focusing effect and subsequent jet (308) generation, we need to secure curved liquid/air interface. That is realized with a gas channel (305) allowing bubble (306) creation at an outing of the gas channel (305) in the fluid (304) of the nozzle. Laser radiation (303) is delivered by an optical waveguide (302), e.g., an optical fiber, and it is in the direct contact with the fluid (304) which is capable of absorption in the spectrum of the laser pulse radiation (303). The FIG. 3b shows a drawing of a jet (308) emerging after the energy of the laser pulse (303) is absorbed. Absorption leads to a rapid vaporization of the fluid (304) and generation of the bubble (307). As the bubble (307) expands it pushes the liquid out of the nozzle. Thanks to the concave shape of the liquid/air interface formed by the bubble (306) the jet (308) emerges from the bubble (306) inner interface and it is further accelerated by the flow focusing effect. In one example of the application, the jet velocity is sufficient to penetrate into the substrate (309). In another example of the application the jet velocity is sufficient to pierce through the substrate (309). In yet another example of the application the jet is deposited on the top of the substrate (309) and does not penetrate in the substrate.

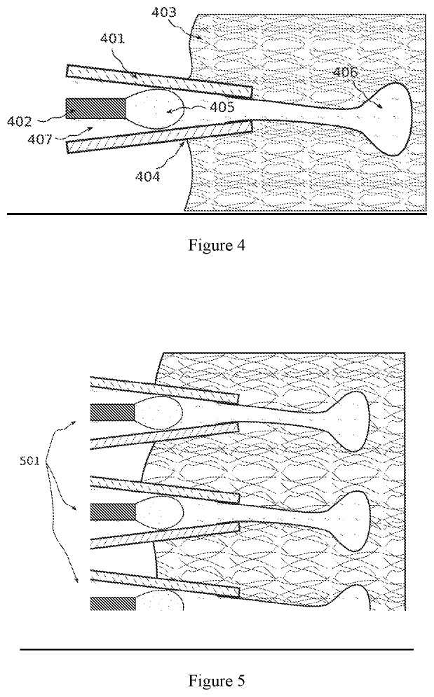

In FIG. 4 is the schematic depiction of the needle assisted jet injection. A substrate (403), for example a tissue, is pierced (404) by a needle (401). The needle (401) is equipped with a pressure generating mechanism (402). Pressure can be generated by the rapid phase transition of a fluid (407) initiated by for example, but not exclusively, laser radiation or an electrical discharge. The created bubble (405) expands and pushes the fluid (407) out of the needle (401) in the substrate (403). Fluid (407) may be deposited in a certain depth (406) depending on the energy of the actuation.

In FIG. 5 is a schematic example of an arrangement consisting of multiple systems described in the FIG. 4 (501) to concurrently perform needle-assisted jet injection. Parallel embodiment of nozzles can be arranged in a pattern.

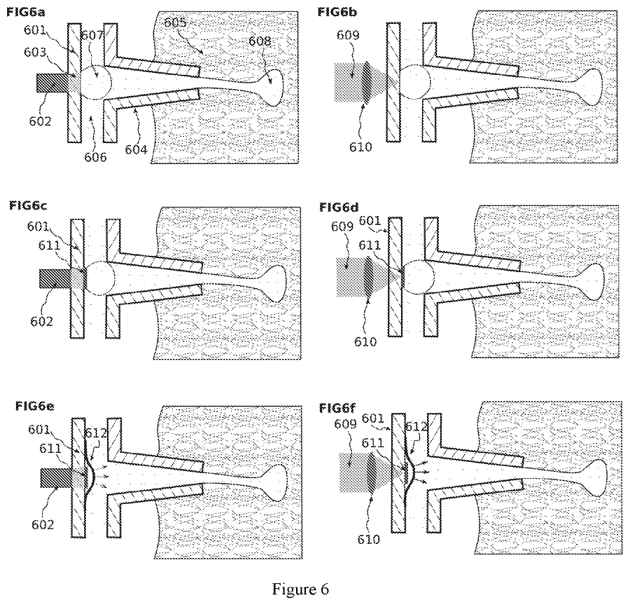

In FIG. 6a is a schematic view of a needle assisted jet injection system. The substrate (605), for example a tissue, is pierced by a needle (604). The needle (604) is equipped with a pressure generating mechanism (602), for example a phase transition initiated by a laser radiation (603). The source of radiation is comparted by a separation (601) transparent to the radiation (603) and it is not in direct contact with the fluid (606). The created bubble (607) expands and pushes the fluid (606) out of the needle (604) in the substrate (605). Fluid (606) may be deposited in a certain depth (608) depending on the energy of the actuation.

FIG. 6b presents the scheme of a modification of the system from the FIG. 6a. In this modification the radiation (609) is focused by a focusing element (610) for example a lens, and passes through a transparent separation (601). It is further absorbed by the fluid (606) which is capable of absorption of the radiation (609).

FIG. 6c presents the scheme of a modification of the system from the FIG. 6a. In this modification the radiation is delivered by an optical waveguide (602) and it is further absorbed by the solid-state light-absorbing material (611) coated on the transparent separation (601). Absorption leads to phase change in the adjacent fluid (606) and generation of the vapor bubble (607).

FIG. 6d presents the scheme of a modification of the system from the FIG. 6a. In this modification the radiation (609) is focused by the focusing element (610), for example the lens, and passes through a transparent separation (601). It is further absorbed by the solid-state light-absorbing material (611) coated on the transparent separation (601). Absorption leads to phase change in the adjacent fluid (606) and generation of the vapor bubble (607).

FIG. 6e presents the scheme of a modification of the system from the FIG. 6a. The source of radiation is delivered by an optical waveguide (602) and is further absorbed by the solid-state light-absorbing material (611) coated on the transparent separation (601). Elastic coating of e.g., polyimide (612) contains the expanding metallic vapor within the blister (617). Deformation of the blister pushes the liquid out of the needle (604) in the substrate (605).

FIG. 6f presents the scheme of a modification of the system from the FIG. 6a. In this modification the radiation (609) is focused by the focusing element (610) for example the lens, and passes through a transparent separation (601); further it is absorbed by the solid-state light-absorbing material (611) coated on the transparent separation (601). Elastic coating of e.g., polyimide (612) contains the expanding metallic vapor within the blister (617). Deformation of the blister pushes the liquid out of the needle (604) in the substrate (605).

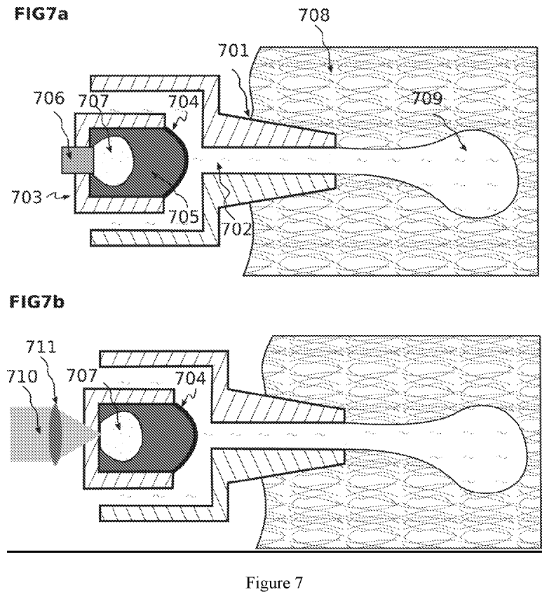

In FIG. 7a is a schematic view of a needle assisted jet injection system using electromagnetic radiation as a driving force for the liquid jet actuation. The substrate (708), for example a tissue, is pierced by a needle (701). The needle (701) is equipped with a pressure generating mechanism (706), for example phase transition initiated by a laser radiation. System consists of two separate compartments; one (703) for the light absorption driving fluid (705) and the second is the channel (702) for the fluid content. Phase changing mechanism (706) for example laser radiation generates a bubble (707). Expansion of the bubble (707) leads to a fast deformation of the elastic membrane (704) which pushes the liquid contain out of the needle (701) deeper (709) into the substrate (708).

FIG. 7b presents the scheme of a modification of the system from the FIG. 7a. In this modification the radiation (710) is focused by an optical focusing element (711) into the chamber containing the driving fluid (705). Absorption leads to the phase transition and subsequent bubble generation (707). Expansion of the bubble (707) leads to a fast deformation of the elastic membrane (704) which pushes the liquid contain out of the needle (701) into the substrate (708).

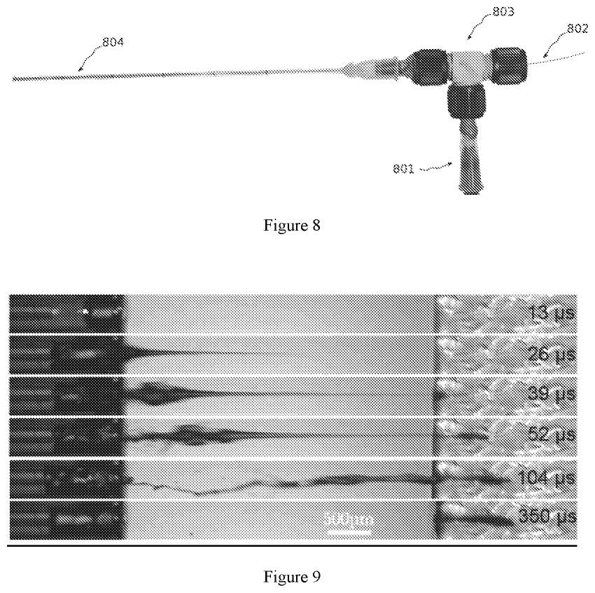

FIG. 8 shows an example of the device using optical pulse as the actuation mechanism for generation of the liquid jet in the air environment. A laser pulse generator (5 ns, 532 nm, Nd:YAG) is coupled into the optical fiber (802). A fluid and the optical fiber (802) channel are connected by a T-junction (803) which is further plugged into a round glass capillary (804). The length of the capillary in this particular example is 95 mm and the outer diameter is 1.2 mm.

FIG. 9 shows an image sequence from an ultrafast camera of the generated liquid jet from the device depicted in the FIG. 8 and its penetration into a gel mimicking mechanical properties of human body soft tissues. A diameter of the optical fiber is 50 μm, capillary inner diameter is 300 μm, the pulse energy is 220 μJ, jet velocity is 140 m/s.

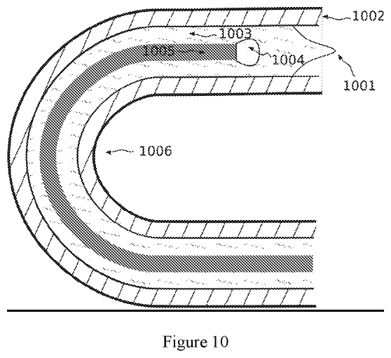

In the FIG. 10 is a schematic view of a system with a flexible tubing. The liquid jet (1001) is generated from a nozzle opening (1002) by means of optically induced cavitation (1003). Liquid and the fiber are embarked in a flexible tubing (1004).

1-34. (canceled)

35. A jet ejection device for jet ejection of a fluid towards a substrate, comprising:
a nozzle having an opening; and
an optical fiber arranged inside the nozzle, the optical fiber configured to provide pulsed radiation into the nozzle,
wherein the nozzle is configured to be filled with the fluid such that the optical fiber is in direct contact with the fluid,
wherein the fluid is configured to absorb at least a part of the pulsed radiation at a first position inside the nozzle to generate a bubble by vaporizing the fluid, and
wherein the nozzle is configured such that an effect of expansion of the bubble inside the nozzle causes a second portion of the fluid to be pushed to the opening of the nozzle to enable the jet ejection of the second portion of the fluid towards the substrate.

36. The jet ejection device of claim 35, wherein the fluid includes an absorbing additive that is configured to improve the absorption of the pulse in the fluid and the vaporization of the fluid.

37. The jet ejection device of claim 35, further comprising:
a focusing mechanism in operative connection with the optical fiber configured to focus the pulsed radiation exiting the optical fiber to an increase of an intensity of the pulse.

38. The jet ejection device of claim 35, further comprising:
a layer of hydrophilic material arranged at the opening of the nozzle, the layer of hydrophilic material configured to enable a formation of a concave meniscus directed to an inner volume of the nozzle, the concave meniscus defining an interface between the fluid and a gaseous environment at the opening of the nozzle, the concave meniscus being configured to center the jet ejection in the nozzle and accelerate the jet ejection by a flow focusing effect.

39. The jet ejection device of claim 38, further comprising
a gas channel configured to deliver gas at the opening of the nozzle to enable the formation of the concave meniscus.

40. The jet ejection device of claim 35, further comprising:
a flexible tubing leading to the nozzle.

41. A needle assisted jet injection device for injection of a fluid to a substrate comprising:
an injection needle having an entrance side through which the fluid can enter the injection needle;
a fluid supply channel configured to supply the fluid, the entrance side of the injection needle attached to an output of the fluid supply channel; and
a pressure generating mechanism configured to apply pressure to the fluid at the entrance side of the injection needle, the pressure generating mechanism including
a pulsed energy supply configured to deliver a pulse of energy to the fluid in a vicinity of the entrance side to cause a rapid phase transition of a first portion of the fluid to thereby generate a bubble configured to expand and inject a second portion of the fluid through the injection needle.

42. The needle assisted jet injection device of claim 41, wherein the pulsed energy supply includes a pulsed radiation source.

43. The needle assisted jet injection device of claim 41, wherein the pulsed energy supply includes an electrical discharge source.

44. A patterned arrangement of a plurality of jet ejection devices that are arranged for jet ejection of a fluid towards a substrate, each jet ejection device comprising:
a nozzle having an opening; and
an optical fiber arranged inside the nozzle, the optical fiber configured to provide pulsed radiation into the nozzle,
wherein the nozzle is configured to be filled with the fluid such that the optical fiber is in direct contact with the fluid,
wherein the fluid is configured to absorb at least a part of the pulsed radiation at a first position inside the nozzle to generate a bubble by vaporizing the fluid, and
wherein the nozzle is configured such that an effect of expansion of the bubble inside the nozzle causes a second portion of the fluid to be pushed to the opening of the nozzle to enable the jet ejection of the second portion of the fluid towards the substrate.

45. The patterned arrangement of claim 44, further comprising:
a radiation splitting device configured to generate a plurality of pulsed radiation, each pulsed radiation corresponding to a respective nozzle of the plurality of jet ejection devices.

46. The patterned arrangement of claim 44, wherein the radiation splitting device includes at least one of a beam splitter, a lens microarray, a hologram, a fiber-based splitter, and/or a fiber bundle system.
